(19) United States
(12) Patent Application Publication
(10) Pub. No.: US 2008/0075343 A1
(43) Pub. Date: Mar. 27, 2008

(54) Method for the positionally accurate display of regions of interest tissue

(76) Inventors: Matthias John, Nurnberg (DE); Norbert Rahn, Forchheim (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/726,623

(22) Filed: Mar. 22, 2007

(30) Foreign Application Priority Data
Mar. 23, 2006 (DE) 10 2006 013 476.1

(51) Int. Cl. A61B 5/00

(52) U.S. Cl. 382/131

(57) ABSTRACT

The invention relates to a method and a device for positioned accurately displaying regions of interest tissue in a three-dimensional reconstruction representation derived from a first image dataset previously recorded for a hollow organ in a patient, comprising: recording catheter image dataset by an image recording catheter placed in the hollow organ and registering the first image dataset with the catheter image dataset; segmenting from the first image dataset a section of interest tissue or a tissue bounding this section and locating the section of tissue; forming an image dataset for the section of tissue using the segmentation and the registration in cropping out from the catheter image dataset the image data which shows this section of tissue; generating an image display of the section of tissue or the region of tissue derived from it and displaying in the three-dimensional reconstruction representation.

### CROSS REFERENCE TO RELATED APPLICATIONS

This application claims priority of German application No. 10 2006 013 476.1 filed Mar. 23, 2006, which is incorporated by reference herein in its entirety.

### FIELD OF THE INVENTION

The invention relates to a method for displaying regions of tissue which are of interest, positioned accurately in a three-dimensional reconstruction representation derived from a first image dataset previously recorded, for a hollow organ in a patient.

### BACKGROUND OF THE INVENTION

Ablation can be undertaken for instance for the treatment of heart rhythm disorders. In doing this, an ablation catheter is introduced, as applicable, into the heart or the region of the heart to be treated, and selective regions of tissue are cauterized by high-frequency currents. It is usual, for the purpose of navigation, to carry out image monitoring by the continuous recording of catheter images. In doing this, the ablation catheter itself can serve as the image recording catheter, or a further image recording catheter can be introduced. The best-known technique for recoding catheter images is intracardial echography (ICE), an ultrasound technique.

If the tissue is cauterized in a particular place, this is referred to as a lesion. Such cauterized areas of tissue are not visible in preoperative first image datasets, this being true not only for ablations have yet to be carried out but also for any lesions which resulted from earlier treatment. It is important for an electro-physiologist, on the one hand, to know the precise location of the lesions in the heart and, on the other hand, to be able to check whether a lesion has been created in full as intended. These regions of tissue which are of interest, in other words the lesions, can basically be seen in the catheter images recorded during the intervention, but it turns out that assignment or segmentation is impossible without further data.

Similar problems arise in the examination or treatment, as applicable, of other hollow organs.

### SUMMARY OF THE INVENTION

The object underlying the present invention is therefore to specify a method with which it is possible to display with positional accuracy regions of tissue which are of interest, together with high-resolution anatomical data, during invasive procedures in hollow organs.

For the purpose of achieving this object, provision is made for the following steps to be carried out during a method of the type indicated in the introduction:
a) recording of a three-dimensional catheter image dataset by means of an image recording catheter placed in the hollow organ, where the coordinate systems of the first image dataset and of the catheter image dataset are in register with each other or are brought into register with each other,
b) segmentation from the first image dataset of a section of tissue which includes the tissue of interest, or of a tissue bounding this section, and localization of the section of tissue,
c) for the purpose of forming an image dataset for the section of tissue, using the segmentation and the registration in cropping out from the catheter image dataset the image data which shows this section of tissue
d) generation of an image display of the section of tissue, or of the region of tissue derived from it which is of interest, and its display in a three-dimensional reconstruction representation.

In this way, the image recording catheter with which the catheter image dataset is recorded can at the same time be an interventional catheter, or alternatively an additional catheter, if an invasive procedure is to be immediately undertaken. The registration of the image datasets can be performed in two ways. First, the registration can be effected before the catheter image dataset is recorded, by an adjustment of the coordinate systems of the image recording catheter and a modality for the recording of the first image dataset, on the basis of a known location relationship for the coordinate systems. This is a simple possibility if, for example, both modalities are part of a single examination and treatment facility, for which a global coordinate system is defined ab initio. If necessary, a calibration can then also take place to adjust the coordinate systems to one another, so that the catheter images are recorded directly in the same coordinate system as that of the first image dataset. As an alternative to this, it is also possible to achieve the registration after the catheter image dataset has been recorded, using anatomical structures or marked points which can be recognized in both image datasets. Such registration methods, for registering the two coordinate systems after the recordings are made, are generally known.

In accordance with the invention a section of tissue including the tissue of interest, or a tissue bounding this section, is then segmented out from the first image dataset. Various segmentation methods for the purpose of automatic segmentation, for example a threshold-based segmentation or a so-called “region growing” segmentation are also conceivable. Segmentation can also take place by the user selecting regions in a display, and by semi-auto-matic procedures in which the user specifies a starting point in a display, in particular for “region growing” segmentation. Here, the section of tissue can be the region of tissue of interest itself, if for example a particular type of tissue is of interest, or can merely include the region of tissue of interest as a subsidiary region.

The localization and segmentation of this section of tissue which has been carried out makes it then possible, because the first image dataset is registered with the catheter image dataset, to select from the catheter image dataset exactly the appropriate regions and to crop them out as separate tissue section image datasets which show the section of tissue in the catheter images. The segmentation and localization of the section of tissue serves, so to speak, to form a mask or template with the help of which the catheter image dataset can be reduced to a dataset for the section of tissue which is actually of interest. As a result, information which is known from the preoperative data can be used advantageously to filter out the image data which is really relevant from the catheter images. This data can then if necessary be further processed, in order to pick out the regions of tissue of interest within it, if the entire section of tissue does not form the region of tissue of interest.

When a image representation has been generated for the section of tissue, or the region of tissue derived from it which is of interest, then it is finally displayed as a three-dimensional reconstruction representation. The display of the section of tissue naturally also shows the region of tissue of interest, because it is included in the section of tissue. The person carrying out the treatment or examination now gets a single display showing all the important items of information, in other words both the anatomy in high-resolution form from the first image dataset and also a positionally accurate representation of the regions of tissue within it which are of interest. The regions of tissue of interest can then, for example, be highlighted in color or incorporated into the display in some other way which makes them distinguishable from the image data of the first image dataset. In doing this, it is advantageous if all the “superfluous” image data from the catheter image dataset is omitted. In this connection it is noted here that it is, of course, also possible in principle to handle several regions of tissue of interest in this way. For example, it is then possible to segment and localize a first section of tissue and a second section of tissue, each of which includes regions of tissue which are of interest, and then to apply the two resulting masks or templates to the catheter image dataset.

The method can be applied with particular advantage in the context of ablation treatments in the heart. In this case, a record can be made of the heart as the hollow organ, and the myocardium considered as the section of tissue. The myocardium is the region of tissue, extending between the endocardium and the epicardium, in which the lesions are produced during ablation treatments and in which they must be produced completely, in accordance with the treatment plan. Various alternatives for segmenting the myocardium are also conceivable. Thus, the endocardium could be segmented from the first image dataset, with the myocardium being defined as surrounding the endocardium to a predetermined thickness, with the blood possibly containing a contrast agent. Here then, a tissue bounding the section of tissue is segmented out from the first image dataset. The myocardium has a thickness which is essentially uniform over a large region, so that such an assumption leads to results which are usable in practice. In another alternative, the myocardium can be segmented directly. In doing this it is particularly advantageous if a contrast agent which accumulates in the myocardium is injected, in order to simplify the segmentation procedure. As a third and final possibility, the endocardium and the epicardium can be segmented, with the myocardium being defined as the region lying between them. By this means again, the position and extent of the myocardium is determined exactly. Finally, parts of the segmentation or the complete segmentation can also be effected manually. For this purpose, the first image dataset is displayed to the user who, for example, either selects a starting point for a “region growing” segmentation or marks the complete myocardium, by which means it is localized. In the last step of the method, irrespective of how the myocardium has been localized, either the myocardium itself is displayed in the three-dimensional reconstruction representation, with the visible lesions (section of tissue with the regions of tissue which are of interest), or the lesions alone (regions of tissue which are of interest).

In doing this, it is of course not only those lesions created during the current intervention which are displayed, but also lesions from any past ablation procedure. If recordings of the past ablation procedure are also available, from which the older lesions can be identified and localized, then those lesions which have already been produced in the current intervention can be shown specially identified in the high-resolution three-dimensional reconstruction representation.

The lesions which arise in the heart during the ablation are only a special case of anomalies which can make up the regions of tissue which are of interest in terms of the present invention. As regions of tissue which are of interest these anomalies, in particular the lesions, can now advantageously be extracted by reference to the image data set for the section of tissue, and displayed in the three-dimensional reconstruction representation. It generally only by selecting the image data set for the section of tissue that an effective and reliable extraction of the anomalies can be achieved.

For the purpose of extracting these anomalies out from the image data set for the section of tissue, several effective alternatives are conceivable. On the one hand, the anomalies, in particular the lesions, can be extracted automatically using, in particular, a threshold-based segmentation method. As the starting point for this, or as an alternative to it, a user can mark the anomalies, in particular the lesions, as a starting point for the extraction in a display output on a monitor of the image data set for the section of tissue. This is particularly helpful for so-called “region growing” segmentations. As an alternative to these possibilities the anomalies, in particular the lesions, can be completely marked up by a user, in a display of the image data set for the section of tissue, and the marked items extracted. In doing this, the user then utilizes his available technical knowledge to localize the anomalies, in particular the lesions, as accurately as possible in the image data set for the section of tissue.

This extraction of the anomalies avoids additional superfluous data, so that the person carrying out the examination or treatment is given only the regions of tissue which really are of interest to them, the anomalies, as supplementary elements in the three-dimensional reconstruction representation. It is possible to recognize at a glance exactly where the anomalies lie, in particular the lesions.

Several advantageous possibilities can be conceived for the ultimate display as a three-dimensional reconstruction representation of the image data for the section of tissue, or for the regions of tissue derived from it which are of interest. Thus, the image data for the section of tissue, or the regions of tissue derived from it which are of interest, can be shown by projection onto a boundary of the section of tissue, in particular the endocardium. Particularly suitable for this purpose is the use of a “maximum intensity projection” method. In the case when the heart is the hollow organ, a three-dimensional image from the inside can be generated, with extracted lesions simply being merged onto the epicardium, and being immediately recognizable. Alternatively or additionally, the appropriate region of the image dataset for the tissue section, or the regions of tissue derived from it which are of interest, can be shown overlaid on a cross-section through the section of tissue, in particular the myocardium. In doing so it is appropriate to use another color or another graphic rendition, so that the viewer can more easily distinguish between the items of data and thus obtain the desired information more quickly. From such a section it is also possible, in particular, to extract depth information.

In particular, the display can be made as a “fly” visualization or by “volume rendering”. The “volume rendering” technique (VRT) permits a view from outside onto the hollow organ, “fly” visualization a view from inside.

Normally, the three-dimensional catheter image dataset can be reconstructed from two-dimensional catheter images. In doing this it is expedient, for the purpose of the reconstructing the catheter image dataset, to make use of the location and orientation data from a location and navigation system. Such a location system can also be used advantageously for the purpose of calibration during registration of the coordinate systems.

The method can be carried out to particular advantage in real time. By this means it is possible, for example during an ablation procedure, continuously to check and monitor the correct position and completeness of the lesions. The doctor can thereby follow exactly where the lesions are developing, and in real time, and adjust the subsequent course of the procedure according to this highly exact data.

The catheter image dataset often also contains further data which, in the case of a real-time display, can advantageously be introduced into the three-dimensional reconstruction representation. Thus it is possible, for example, to extract from the catheter image dataset an intervention catheter, in particular an ablation catheter, and to display it in the three-dimensional reconstruction. The person carrying out the treatment or examination can thus, for example during an intervention in the heart, exercise effective control of the ablation catheter, for example to enable the finishing of incomplete scleroses i.e. lesions.

In doing this, it can be expedient to use as the image recording catheter an ultrasonic image recording catheter, in particular an ICE catheter. The first image dataset can be a computer tomography image dataset or a magnetic resonance image dataset. However, in the context of this method it is also possible to use other recording modalities.

In closing, attention should be called to the fact that in the case of hollow organs which are affected by the heart cycle or breathing cycle, obviously within the framework of the method only image datasets associated with the same ECG or breathing phase should be processed together. To this end, a known method, for example for ECG or breathing triggering, can be carried out. Alternatively, the ECG phase can be recorded for each image, and images with the same phase can be jointly subject to further processing.

### BRIEF DESCRIPTION OF THE DRAWINGS

Further advantages and details of the present invention are evident from the exemplary embodiment described below and by reference to the drawings. These show:

FIG. 1 a medical examination facility in which the method in accordance with the invention can be performed,

FIG. 2 a flow diagram of the method in accordance with the invention,

FIG. 3 an outline of the principle, explaining the steps in the method,

FIG. 4 display of a section through the myocardium, with the lesions merged onto it, and

FIG. 5 an outline of the principle for the “maximum intensity projection”.

### DETAILED DESCRIPTION OF THE INVENTION

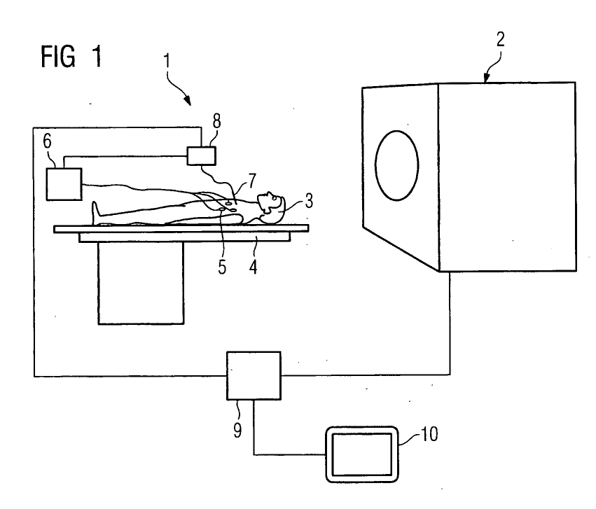

FIG. 1 shows a medical examination facility 1. In this, heart ablation procedures can be undertaken. For this purpose, the first step is the preoperative recording of a first image dataset in a computer tomography facility 2, from which a three-dimensional reconstruction representation of the heart can be obtained. During the actual operation, a patient 3 is positioned on a patient bed 4. An ECG measuring device 6 monitors the heart cycle via a suitable system of sensors 5. A catheter 7 is introduced into the patient's heart. It incorporates an ablation device together with an image recording device, and is actuated via a catheter controller 8. The link between the ECG measuring device 6 and the catheter controller 8 enables ECG-triggered images to be recorded.

A similar triggering device is provided and can be used for the computer tomography facility 2. Using the image recording device of the catheter 7, a catheter image dataset can be recorded in real time during an intervention. The catheter images, together with any ECG data from the ECG measuring device 6, are passed on from the catheter controller 8 to a computation facility 9, in which is already stored the image data for a first image dataset, recorded in the computer tomography facility. A monitor 10 is used for displaying image data. The computation facility 9 is now constructed so that, using data from the first image dataset, it extracts in real time the myocardium, or the lesions it contains, and displays them as high-resolution anatomy accurately positioned in a three-dimensional reconstruction representation of the first image dataset.

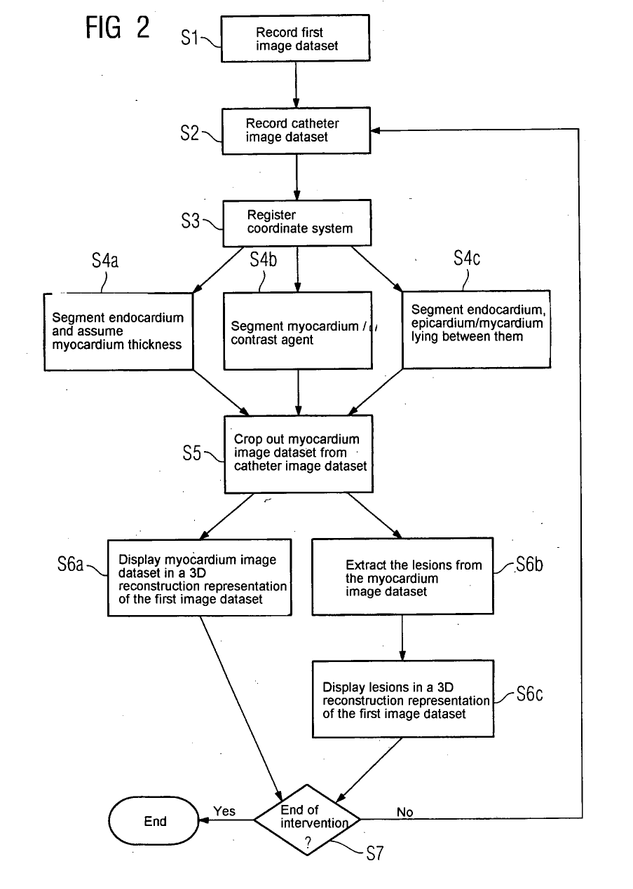

FIG. 2 shows a flow diagram of the method in accordance with the invention, as it can be carried out in real time using the medical examination facility 1.

First, in step S1 a first image dataset is recorded by means of the computer tomography facility 2. During the intervention, a catheter image dataset is then recorded by means of the image recording device in the catheter 7, here an ICE device, this being ECG-triggered in such a way that the ECG phase of the catheter image dataset corresponds to the ECG phase of the first image dataset. In doing this, two-dimensional cross-sectional images are initially recorded, from which the three-dimensional catheter image dataset is reconstructed using the computation facility 9, or even in the catheter controller itself 8.

In step S3, the coordinate systems, i.e. that of the first image dataset and that of the catheter image dataset, are registered with each other. For this purpose, generally known methods of registration can be used. If there is already a global coordinate system defined in the medical examination facility 1, against which the computer tomography facility 2 or the catheter 7, as applicable, can be calibrated, then this calibration can be carried out even before the recording of the catheter image dataset is carried out in step S2. In such a case, step S3 would be omitted.

The purpose of step S4 is now to localize the region of the first image dataset in which the myocardial tissue is located, where the lesions are to be created or have been created, as applicable. The myocardium itself can only with difficulty be recognized in the ICE recording from the catheter 7, so that there are ultimately three possibilities for localizing it, these alternatives being shown in FIG. 2 as the steps S4a, S4b and S4c.

In a first alternative, step S4a, the endocardium is first segmented. The endocardium is really easy to find, because it separates the blood mass from the tissue, with there possibly being a contrast agent in the blood. Since the myocardium adjoins the endocardium, and has a very uniform thickness, a region around the endocardium with a fixed thickness of, for example, 5 mm is defined as the region in which the myocardium has been localized.

Another possibility for localizing the myocardium is provided by the administration of a contrast agent which accumulates in the myocardium and is visible in the first image dataset. When such a contrast agent is used, it is possible to segment the myocardium directly, cf. step S4b.

The third alternative is the segmentation of the endocardium and the epicardium. These two regions of tissue enclose between them the myocardium, so that the region in which the myocardium is located is the region lying between the epicardium and the endocardium.

Obviously, such a segmentation can in principle also include manual involvement by a user, or can be carried out entirely manually by a user.

By this means it is now known where the section of tissue which is initially being sought, the myocardium, is located in the coordinate system of the first image dataset, which is indeed registered with the coordinate system of the catheter image dataset. The corresponding region in the catheter image dataset—easy to determine via the registration—which consequently also shows the myocardium, can now be cropped out from the catheter image dataset. This takes place in step S5. The region into which the myocardium has been localized is thus in effect overlaid on the catheter image dataset like a mask or template, and only the regions of this image dataset within this mask or template are given further consideration. This remaining part of the catheter image dataset is the myocardium image dataset. As a result, only the catheter image data from the myocardial tissue is examined further, because this is where the lesions which are ultimately being sought will be found.

There are now once again two possible ways for the method to continue. One possibility is the direct display of the myocardium image dataset in a 3D reconstruction representation of the first image dataset, step S6a. The image data for the myocardium image dataset is incorporated, possibly in another color or identified in some other way, into the anatomy of the three-dimensional reconstruction representation of the first image dataset, accurately positioned and correctly detailed. Using the ICE data which can be seen in addition, an experienced doctor can now recognize the lesions in the image and assess their position, orientation and completeness, in order to then determine how to continue the procedure.

Alternatively, however, it is also possible, to extract the lesions from the myocardium image dataset, step S6b. This can be done automatically, using a segmentation method, but also semi-automatically or by the user himself. If the user is involved, then the myocardium image dataset is displayed on the monitor 10, and the user can specify a start point for the segmentation or even mark the lesions in their entirety. They are then extracted, which means either that against a voxel can be simply stored whether there is a lesion at that point (binary: “yes” or “no”). Or alternatively, the myocardium image dataset can be further “cut”, in that only the image data for those regions which contain lesions is retained. In any case, a lesion image dataset results. This too is now included in a display, step S6c, of a three-dimensional reconstruction representation of the first image dataset, so that the user or doctor, as applicable, can make appropriate decisions.

When the intervention is over, step S7, then the method also ends, it being obviously possible to save the image datasets obtained for later checking or further examination. If the intervention is continued, then the method starts again in step S2 with the recording of a new catheter image dataset, to make a real time display possible. The doctor can thus watch the change in the heart tissue arising from the interventions.

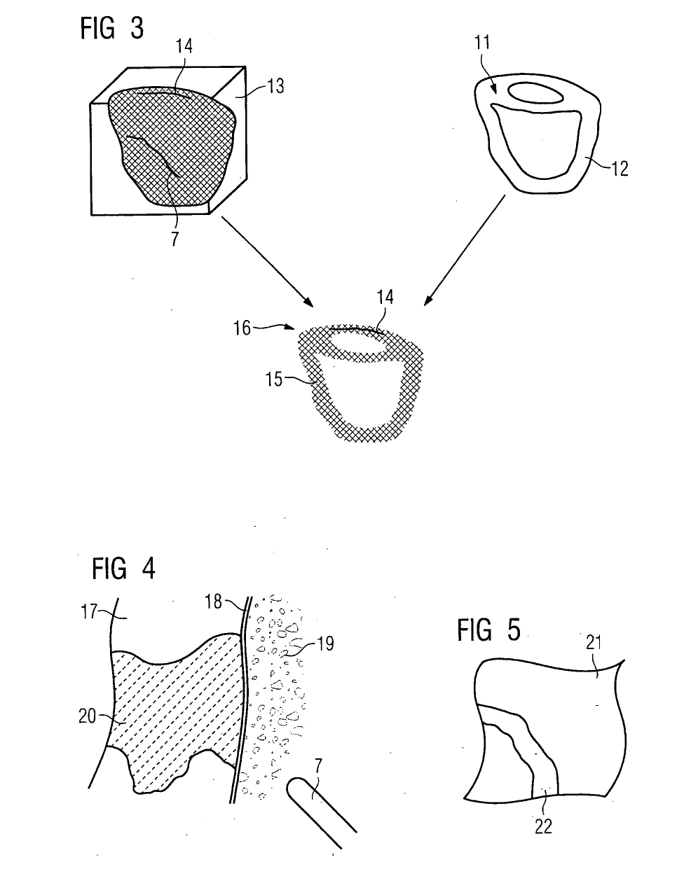

FIG. 3 shows more precisely, in the form of a schematic diagram, how the image dataset for the section of tissue, here the myocardium image dataset, is obtained using the method in accordance with the invention. Reference mark 11 shows the localization of the myocardium 12, obtained from the three-dimensional first image dataset, determined by appropriate segmentation in steps S4a, S4b or S4c. At the same time, a catheter image dataset 13 is available, in which the myocardium itself is not precisely identifiable, although it is possible to recognize what is presumably a lesion 14 and the catheter 7 in the catheter image dataset 13. The location data for the myocardium 12 is now overlaid on the catheter image dataset like a template, and only the regions 15, in which the myocardium can be seen in the catheter image dataset 13, are examined further. This produces the myocardium image dataset 16. Evidently, the lesion 14 really is a lesion because it is located in the myocardium. The lesion 14 can now, for example—cf. step 6b—be further extracted.

At this point it is noted that because the coordinate systems of the catheter image dataset and the first image dataset are in any case registered, the location data which can be obtained about the catheter 7 from the catheter image dataset 13 can also be expediently determined, in order to incorporate the position of the catheter 7, again with high precision, into the real time display of the three-dimensional reconstruction representation of the first image dataset and of the lesions or the myocardium.

In the method according to the invention, there are various possibilities for the display. Using the “volume rendering” technique (VRT), a three-dimensional view of the heart from outside can be produced. “Fly” visualization permits a view from inside.

The display of the lesions or the myocardium, as applicable, in the three-dimensional reconstruction representation can be effected simply by overlaying. Two display options are explained below in more detail.

FIG. 4 shows a cross-sectional view through the myocardial tissue 17. On the inner side of the heart, the myocardial tissue 17 is bounded and separated from the blood 19 by the endocardium 18. By a change of color or darkening, an extracted lesion 20 is included in the display by overlaying it onto the image data for the first image dataset. This cross-sectional view gives one precise depth information about the lesion 20, in an advantageous manner. In addition the catheter 7 which is located in this section is also shown in the cross-sectional view.

However, it is also possible, in particular in the “fly” visualization, to project the data about the myocardium or the lesion, as applicable, for example onto a surface, in particular the endocardium. For this purpose it is possible to use, for example, the “maximum intensity projection” method. With this, the voxel which has the highest value is projected onto the endocardium along a line which is perpendicular to or in a defined direction relative to the surface of the endocardium and goes backward into the myocardium. This results in the depth data in the sectional view of FIG. 4 being lost, but makes possible a three-dimensional view which is simple to interpret. As an example of this, FIG. 5 shows an extract from the surface of the endocardium 21. Projected onto this at 22 can be seen a lesion.

1.-18. (canceled)

19. A method for positioned accurately displaying a section of an interest tissue of a hollow organ of a patient in a three-dimensional reconstruction representation derived from a previously recorded first image dataset, comprising:
generating a catheter image dataset by an imaging recording catheter placed in the hollow organ;
registering a coordinate system of the first image dataset with a coordinate system of the catheter image dataset;
segmenting the section of the interest tissue from the first image dataset;
cropping out an image data showing the section of the interest tissue from the catheter image dataset based on the registration and the segmentation; and
displaying the section of the interest tissue in the three-dimensional reconstruction representation.

20. The method as claimed in claim 19, wherein a tissue bounding the section is segmented from the first image dataset.

21. The method as claimed in claim 19, wherein the hollow organ is a heart of the patient and the section of the interest tissue is a myocardium of the heart.

22. The method as claimed in claim 21, wherein the myocardium is segmented from the first image dataset by:
segmenting an endocardium of the heart from the first image dataset and defining the myocardium as a region surrounding the endocardium to a predefined depth, or
segmenting the myocardium based on a contrast agent accumulated in the myocardium, or
segmenting the endocardium and an epicardium of the heart and defining the myocardium as a region between the endocardium and the epicardium.

23. The method as claimed in claim 19, wherein a region of the interest tissue is extracted from the image dataset of the section of the interest tissue and is displayed in the three-dimensional reconstruction representation.

24. The method as claimed in claim 23, wherein the region of the interest tissue is extracted automatically and the automatic extraction is based on a threshold segmentation method.

25. The method as claimed in claim 23, wherein the region of the interest tissue is an anomaly or a lesion in the section of the interest tissue.

26. The method as claimed in claim 25, wherein a user marks the anomaly or the lesion in the display of the section of the interest tissue as a starting point for the extraction.

27. The method as claimed in claim 25, wherein a user completely marks the anomaly or the lesion in the display of the section of the interest tissue and the marked item is extracted.

28. The method as claimed in claim 19, wherein a boundary of the section of the interest tissue is projected in the three-dimensional reconstruction representation and the projection is a maximum intensity projection.

29. The method as claimed in claim 19, wherein the section of the interest tissue is overlaid on a cross-section through the section of the interest tissue in the three-dimensional reconstruction representation.

30. The method as claimed in claim 19, wherein the section of the interest tissue is displayed by a fly visualization or by volume rendering.

31. The method as claimed in claim 19, wherein the catheter image dataset is a three-dimensional catheter image dataset and is reconstructed from a plurality of two-dimensional catheter images recorded by the image recording catheter.

32. The method as claimed in claim 31, wherein the three-dimensional catheter image dataset and is reconstructed based on a location and an orientation data of the image recording catheter obtained from a location and navigation system connected to the image recording catheter.

33. The method as claimed in claim 19, wherein the image recording catheter is an ultrasonic image recording catheter and the ultrasonic image recording catheter is an ICE catheter.

34. The method as claimed in claim 19, wherein the section of the interest tissue is displayed in the three-dimensional reconstruction representation in a real time.

35. The method as claimed in claim 19,
wherein an intervention catheter is extracted from the catheter image dataset and is displayed in the three-dimensional reconstruction, and
wherein the intervention catheter is an ablation catheter.

36. The method as claimed in claim 19, wherein the first image dataset is a computer tomography image dataset or a magnetic resonance image dataset.

37. The method as claimed in claim 19, wherein the section of the interest tissue is segmented based on a threshold or region growing.

38. A medical device for positioned accurately displaying a section of an interest tissue of a hollow organ in a patient in a three-dimensional reconstruction representation derived from a previously recorded first image dataset, comprising:
an image recording catheter placed in the hollow organ that records a catheter image dataset;
a computation device that:
registers a coordinate system of the first image dataset with a coordinate system of the catheter image dataset,
segments the section of the interest tissue from the first image dataset,
crops out an image data showing the section of the interest tissue from the catheter image dataset based on the registration and the segmentation; and
a monitor that displays the section of the interest tissue in the three-dimensional reconstruction representation.
